(19) United States
(12) Patent Application Publication
(10) Pub. No.: US 2023/0191196 A1
(43) Pub. Date: Jun. 22, 2023

(54) INFORMATION PROCESSING DEVICE, INFORMATION PROCESSING METHOD AND INFORMATION PROCESSING PROGRAM

(71) Applicant: FUJIFILM CORPORATION, Tokyo (JP)

(72) Inventor: Yasuhisa KANEKO, Tokyo (JP)

(21) Appl. No.: 18/065,635

(22) Filed: Dec. 14, 2022

(30) Foreign Application Priority Data
Dec. 21, 2021 (JP) 2021-207604

(51) Int. Cl. A63B 24/00; G16H 20/30

(52) U.S. Cl. CPC A63B 24/0062; G16H 20/30; A63B 2024/0071; A63B 2220/52

(57) ABSTRACT

An information processing device comprising at least one processor, wherein the at least one processor is configured to: acquire a plurality of pieces of exercise information indicating each of a plurality of types of exercise amounts measured according to exercise of a subject; acquire a weight derived in advance based on the exercise information for each type of the exercise information; and derive a load amount applied to a body of the subject based on the plurality of pieces of exercise information and the weight.

### CROSS-REFERENCE TO RELATED APPLICATIONS

The present application claims priority under 35 U.S.C. § 119 to Japanese Patent Application No. 2021-207604, filed on Dec. 21, 2021. The above application is hereby expressly incorporated by reference, in its entirety, into the present application.

### BACKGROUND

### Technical Field

The present disclosure relates to an information processing device, an information processing method, and an information processing program.

### Related Art

In the related art, techniques are known for supporting rehabilitation to improve deterioration in physical function due to trauma, aging, brain dysfunction, and the like.

For example, WO2019/187099A discloses analyzing changes in physical function of a person based on time-series changes in physical condition of the person and presenting suggestions for improving the physical function. Further, for example, WO2020/234957A discloses determining a condition of a user based on sensor data including biological information of the user, predicting whether or not the user will perform rehabilitation in the future, and presenting rehabilitation support information. Further, for example, JP2020-187550A discloses comparing a transition of a biological index value indicating a condition of a subject and a transition of therapeutic means implemented by the subject with past cases and predicting a timing to reach a target biological index value.

In the rehabilitation performed to improve the physical function, a plurality of kinds of exercise training having different types and intensities of exercise may be combined, and it is considered that degrees of effects of various types of exercise training vary according to a state of the physical function of the subject. For example, in the rehabilitation of running ability, the running ability may be improved more effectively by starting from walking training with low intensity in an early stage and performing jogging and dash training with high intensity as the physical function improves. Further, for example, if high-intensity training is performed in the early stage of rehabilitation, the physical function may rather deteriorate due to excessive loads.

Therefore, for example, in the early stage of rehabilitation, it may be effective to perform exercise training while focusing on the number of steps rather than the walking speed, whereas in a late stage of rehabilitation, it may be effective to perform exercise training while focusing on the walking speed rather than the number of steps. As described above, there is a desire for a technique that can support the improvement of the physical function by planning the content of exercise training in the rehabilitation in consideration of the effects of various types of exercise training that vary according to the state of the physical function of the subject.

### SUMMARY

The present disclosure provides an information processing device, an information processing system, an information processing method, and an information processing program that can support the improvement of the physical function.

A first aspect of the present disclosure is an information processing device comprising at least one processor, in which the processor is configured to: acquire a plurality of pieces of exercise information indicating each of a plurality of types of exercise amounts measured according to exercise of a subject; acquire a weight derived in advance based on the exercise information for each type of the exercise information; and derive a load amount applied to a body of the subject based on the plurality of pieces of exercise information and the weight.

In the above aspect, the plurality of pieces of exercise information are information indicating each of a plurality of types of exercise amounts measured over time, and the processor may acquire the weight that varies over time according to a measurement time of the exercise information.

In the above aspect, the processor may acquire a plurality of pieces of learning exercise information that are of the same type as the plurality of pieces of exercise information and that are measured over time, derive the weight that varies over time according to a measurement time of the learning exercise information based on the plurality of pieces of learning exercise information, and derive the load amount based on the weight derived at the measurement time of the learning exercise information corresponding to the measurement time of the exercise information.

In the above aspect, physical information indicating a physical level of the subject being a measurement source at the measurement time is added to the exercise information and the learning exercise information, and the processor may derive the weight based on the learning exercise information to which the physical information corresponding to the physical information added to the exercise information is added.

In the above aspect, the physical level may indicate an improvement level in a case of improving a physical function of the subject.

In the above aspect, the processor may derive a load amount applied to a part of the body of the subject as the load amount.

In the above aspect, the processor may derive the load amount based on the plurality of pieces of exercise information measured within a predetermined period.

In the above aspect, the exercise information may indicate at least one of the number of steps, a walking speed, an electromyogram, or a flexion angle or a flexion speed of a joint of the subject.

A second aspect of the present disclosure is an information processing method comprising: acquiring a plurality of pieces of exercise information indicating each of a plurality of types of exercise amounts measured according to exercise of a subject; acquiring a weight derived in advance based on the exercise information for each type of the exercise information; and deriving a load amount applied to a body of the subject based on the plurality of pieces of exercise information and the weight.

A third aspect of the present disclosure is an information processing program for causing a computer to execute a process comprising: acquiring a plurality of pieces of exercise information indicating each of a plurality of types of exercise amounts measured according to exercise of a subject; acquiring a weight derived in advance based on the exercise information for each type of the exercise information; and deriving a load amount applied to a body of the subject based on the plurality of pieces of exercise information and the weight.

According to the above aspects, the information processing device, the information processing method, and the information processing program of the present disclosure can support the improvement of the physical function.

### BRIEF DESCRIPTION OF THE DRAWINGS

FIG. 1 is a schematic configuration diagram of an information processing system.

FIG. 2 is a block diagram showing an example of a hardware configuration of an information processing device.

FIG. 3 is a block diagram showing an example of a functional configuration of the information processing device.

FIG. 4 is a diagram for explaining a weight derivation method.

FIG. 5 is a diagram for explaining a method of deriving a load amount.

FIG. 6 is a correlation diagram showing an example of a correlation between biological information and a total load amount.

FIG. 7 is a diagram for explaining a biological information prediction method.

FIG. 8 is a diagram showing an example of a screen displayed on a display.

FIG. 9 is a flowchart showing an example of a weight derivation process.

FIG. 10 is a flowchart showing an example of a load amount derivation process.

FIG. 11 is a flowchart showing an example of a prediction process.

FIG. 12 is a flowchart showing an example of a planning process.

### DETAILED DESCRIPTION

Exemplary embodiments for implementing a technique of the present disclosure will be described in detail below with reference to the drawings.

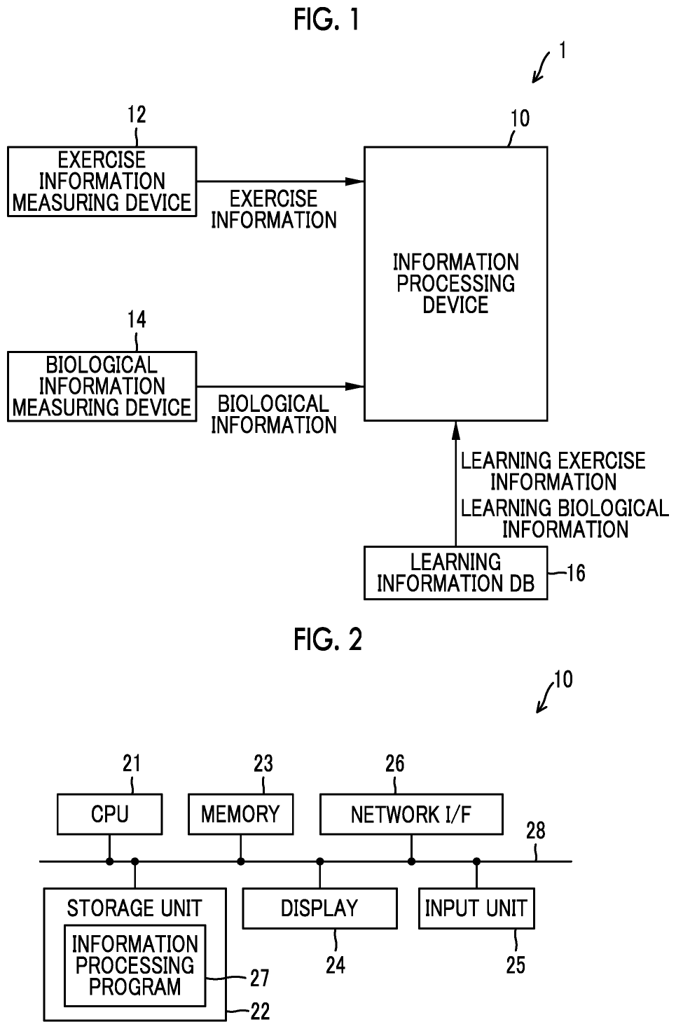

An example of a configuration of an information processing system 1 according to the present embodiment will be described with reference to FIG. 1. As shown in FIG. 1, the information processing system 1 comprises an information processing device 10, at least one exercise information measuring device 12, at least one biological information measuring device 14, and a learning information database (DB) 16. The information processing device 10 and the exercise information measuring device 12, the information processing device 10 and the biological information measuring device 14, and the information processing device 10 and the learning information DB 16 can communicate with each other by wired or wireless communication, respectively.

The exercise information measuring device 12 has a function of measuring exercise information indicating an exercise amount measured according to exercise of a subject. Here, the exercise information indicates an exercise amount measured over time and may be, for example, information indicating at least one of the number of steps, a walking speed, an electromyogram, or a flexion angle or a flexion speed of a joint of the subject. In these cases, as the exercise information measuring device 12, for example, a wearable terminal such as a smartwatch equipped with a sensor capable of detecting exercise such as a pedometer, a myoelectric sensor, and an acceleration sensor can be applied. Also, a plurality of these devices may be combined and applied.

The biological information measuring device 14 has a function of measuring biological information of the subject. For example, the biological information may be information indicating at least one of a body temperature, a heart rate, an electrocardiogram, a myoelectricity, a blood pressure, an arterial blood oxygen saturation (SpO2), a blood glucose level, a lipid level, or the like. In these cases, as the biological information measuring device 14, for example, a wearable terminal such as a thermometer, a heart rate meter, a blood glucose self-measuring device, and a smart watch comprising a sensor for measuring biological information such as a heart rate and an arterial blood oxygen saturation can be applied.

Further, for example, the biological information may be information indicating at least one of a medical image captured by a medical imaging device or a feature amount that can be analyzed from the medical image. The medical imaging device is a device that performs, for example, X-ray imaging, computed tomography (CT), magnetic resonance imaging (MRI), ultrasound diagnostic imaging, endoscopic imaging, fundus imaging, positron emission tomography (PET), and the like. By using these medical imaging devices as the biological information measuring device 14, a medical image as biological information can be obtained. The feature amount is, for example, an information amount indicating a volume, a major diameter, a minor diameter, a pixel value, and the like of an abnormal shadow such as a lesion and a structure to be diagnosed.

Further, for example, the biological information may be information indicating a result of at least one of a hematological test, an infectious disease test, a biochemical test, an immunological test, a genetic test, a bacteriological test, or a urine test. The hematological test is, for example, a test in which a leukocyte count, an erythrocyte count, a hemoglobin concentration, or the like is obtained as a test result. The biochemical test is, for example, a test in which various types of indices related to enzymes, proteins, sugars, lipids, and electrolytes are obtained as test results. The infectious disease test is, for example, a test in which presence or absence of infection of various types of infectious diseases such as an influenza infection and a new type coronavirus infection is obtained as a test result.

The immunological test is, for example, a test in which a detection result of a substance peculiar to a tumor marker, a hormone, an allergy, or the like is obtained as a test result. The genetic test is, for example, a test in which genetic information related to a constitution, a disease, or the like is obtained as a test result by analyzing deoxyribonucleic acid (DNA). The bacterial test is, for example, a test in which a type and an amount of bacteria present in a body, on a surface of the body surface, or the like is obtained as a test result. The urine test is, for example, a test in which urinary sugar, urinary protein, urinary occult blood, or the like is obtained as a test result. In a case where these various types of test results are used as the biological information, for example, a known analyzer that performs analysis using blood, urine, or the like as a subject can be applied to the biological information measuring device 14.

The learning information DB 16 is a database in which learning exercise information and learning biological information are stored. The learning information DB 16 is realized by, for example, a storage medium such as a hard disk drive (HDD), a solid state drive (SSD), and a flash memory.

The learning exercise information and the learning biological information are information of the same type as the above-described exercise information and biological information, respectively, and are actual data measured over time in the rehabilitation performed in the past. That is, the learning exercise information and the learning biological information are information measured over time in a period from a start time (for example, immediately after the treatment) to an end time (for example, a recovery completion time) of the rehabilitation. The learning exercise information and the learning biological information may be information measured from another subject different from a subject to be currently processed.

By the way, in the rehabilitation performed to improve the physical function, a plurality of kinds of exercise training having different types and intensities of exercise may be combined, and it is considered that degrees of effects of various types of exercise training vary according to a state of the physical function of the subject. For example, in the rehabilitation of running ability, the running ability may be improved more effectively by starting from walking training with low intensity in an early stage and performing jogging and dash training with high intensity as the physical function improves. Further, for example, if high-intensity training is performed in the early stage of rehabilitation, the physical function may rather deteriorate due to excessive loads.

Therefore, for example, in the early stage of rehabilitation, it may be effective to perform exercise training while focusing on the number of steps rather than the walking speed, whereas in a late stage of rehabilitation, it may be effective to perform exercise training while focusing on the walking speed rather than the number of steps. As described above, there is a desire for a technique that can support the improvement of the physical function by planning the content of exercise training in the rehabilitation in consideration of the effects of various types of exercise training that vary according to the state of the physical function of the subject.

Therefore, the information processing device 10 according to the present embodiment plans the content of the exercise training in the rehabilitation in consideration of the state of the physical function of the subject. Further, in the process thereof, the load amount applied to the body of the subject is derived in consideration of the state of the physical function of the subject. Hereinafter, a detailed configuration of the information processing device 10 will be described.

First, an example of a hardware configuration of the information processing device 10 according to the present embodiment will be described with reference to FIG. 2. As shown in FIG. 2, the information processing device 10 includes a central processing unit (CPU) 21, a non-volatile storage unit 22, and a memory 23 as a temporary storage area. In addition, the information processing device 10 includes a display 24 such as a liquid crystal display, an input unit 25 such as a keyboard, a mouse, and a button, and a network interface (I/F) 26 that performs wired or wireless communication with the exercise information measuring device 12, the biological information measuring device 14, and an external network (not shown). The CPU 21, the storage unit 22, the memory 23, the display 24, the input unit 25, and the network I/F 26 are connected to each other via a bus 28 such as a system bus and a control bus such that various types of information can be exchanged. As the information processing device 10, for example, a personal computer, a server computer, a tablet terminal, a smart phone, a wearable terminal, or the like can be applied.

The storage unit 22 is realized by, for example, a storage medium such as a hard disk drive (HDD), a solid state drive (SSD), and a flash memory. An information processing program 27 in the information processing device 10 is stored in the storage unit 22. The CPU 21 reads the information processing program 27 from the storage unit 22, develops the information processing program 27 into the memory 23, and executes the developed information processing program 27. The CPU 21 is an example of the processor of the present disclosure.

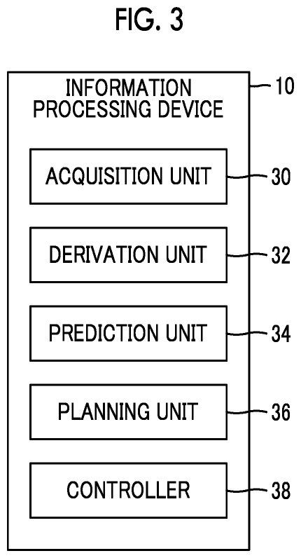

Next, an example of a functional configuration of the information processing device 10 according to the present embodiment will be described with reference to FIG. 3. As shown in FIG. 3, the information processing device 10 includes an acquisition unit 30, a derivation unit 32, a prediction unit 34, a planning unit 36, and a controller 38. By executing the information processing program 27, the CPU 21 functions as the acquisition unit 30, the derivation unit 32, the prediction unit 34, the planning unit 36, and the controller 38.

In the following description, a patient suffering from knee osteoarthritis is assumed to be a subject, and rehabilitation after a treatment (so-called regenerative medicine) in which mesenchymal stem cells are transplanted into a knee cartilage defect to regenerate cartilage is assumed. In addition, information indicating three exercise amounts indicating the number of steps, a walking speed, and a flexion angle of the knee is used as a plurality of pieces of exercise information, and information indicating a defect volume of the knee cartilage analyzed from the MRI image is used as the biological information.

Derivation of Weight

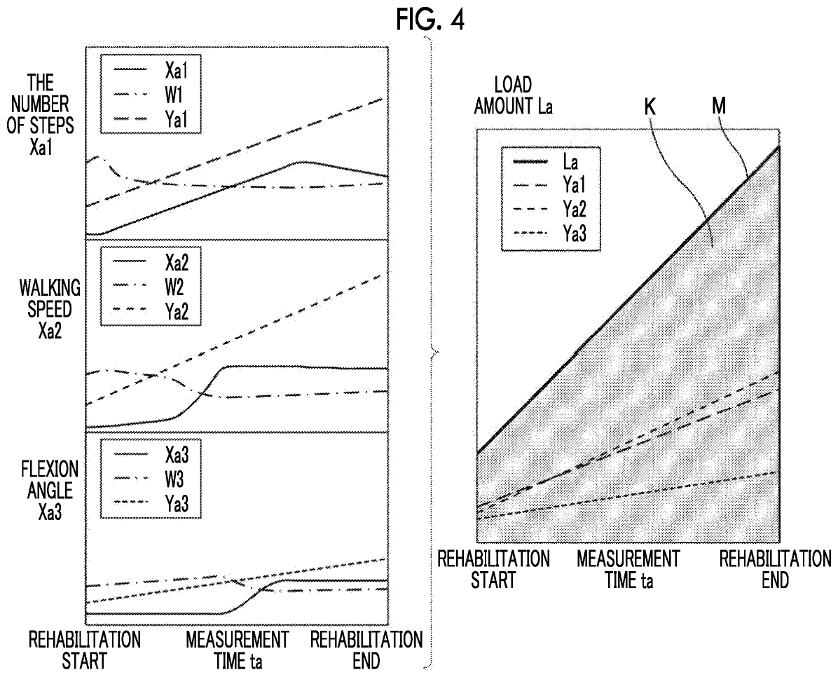

First, a method of deriving the weight for deriving the load amount applied to the body of the subject by the exercise will be described with reference to FIG. 4. The weight is derived based on the learning exercise information for each type of the learning exercise information. In FIG. 4, the number of steps Xa1, a walking speed Xa2, and a flexion angle Xa3 are indicated by solid lines. Further, weights W1, W2, and W3 for each of the number of steps Xa1, the walking speed Xa2, and the flexion angle Xa3 are indicated by one-dot chain lines. Further, a partial load amount Ya1 related to the number of steps Xa1 is shown by a long broken line, a partial load amount Ya2 related to the walking speed Xa2 is shown by a short broken line, and a partial load amount Ya3 related to the flexion angle Xa3 is shown by a dotted line. Further, a load amount La is indicated by a thick solid line.

The acquisition unit 30 acquires a plurality of pieces of learning exercise information measured over time from the learning information DB 16. Specifically, the acquisition unit 30 acquires the learning exercise information that has been measured in the past from a subject having the same disease as that of the subject for whom the content of the exercise training in the current rehabilitation is planned.

It is preferable that the acquisition unit 30 acquires representative learning exercise information that is generated based on a plurality of pieces of learning exercise information measured in the past from a plurality of subjects having the same disease as the target subject for whom the content of the current exercise training is planned. For example, it is preferable that the acquisition unit 30 acquires learning exercise information about each of the plurality of subjects stored in the learning information DB 16 and generates a representative value such as an average value and a median value thereof as learning exercise information used for subsequent processing.

The derivation unit 32 derives a weight Wi(tₐ) that varies over time according to a measurement time of the learning exercise information based on the plurality of pieces of learning exercise information acquired by the acquisition unit 30. A specific derivation method of the weight Wi(tₐ) by the derivation unit 32 will be described using the following calculation equations (1) to (4).

Yai(tₐ)=Wi(tₐ)×Xai(tₐ)×Ci (1)

La(tₐ)=ΣYai(tₐ) (2)

∫La(tₐ)dtₐ=K (3)

dLa(tₐ)/dtₐ=M (4)

Note that i is an integer of 2 or more, and i is assumed to be 1 to 3 in the following description. K and M are positive constants.

Yai(tₐ) in Equation (1) represents a partial load amount applied to the body of the subject by the exercise amount indicated by the learning exercise information Xai(tₐ) at the measurement time tₐ. Ci is a conversion coefficient for converting various types of learning exercise information Xai, such as the number of steps, a walking speed, and a flexion angle, into a partial load amount Yai (that is, aligning units), is determined in advance for each type of the learning exercise information, and is stored in advance, for example, in the storage unit 22.

Wi(tₐ) is a weight that varies over time according to the measurement time tₐ of the learning exercise information Xai(tₐ). As described above, in the rehabilitation, the degree of effect of exercise training with different types and intensities of exercise is considered to vary according to the state of the physical function of the subject. For example, even if the same exercise training is performed at the measurement time tₐ1 in the early stage of the rehabilitation and at the measurement time tₐ2 in the late stage, it is considered that the partial load amount applied to the body of the subject may be different between the measurement times tₐ1 and tₐ2. In other words, even if Xa1(tₐ1) and Xa1(tₐ2) match, Ya1(tₐ1) and Ya1(tₐ2) do not necessarily match. Therefore, in Equation (1), by varying the weight Wi(tₐ) over time, the partial load amount Yai(tₐ) can also be varied over time according to the state of the physical function of the subject.

La(tₐ) shown in Equation (2) is a total sum of the partial load amounts Yai(tₐ) at the measurement time tₐ and represents a total load amount applied to the body of the subject by the exercise amount indicated by various types of learning exercise information Xa1 to Xa3 at the measurement time tₐ. Here, La(tₐ) is set so as to satisfy Equations (3) and (4). Equation (3) means that a time integral of La(tₐ) satisfies a predetermined constant K. Equation (4) means that a time differential of La(tₐ) satisfies a predetermined constant M, that is, La(tₐ) is represented by a straight line of a slope M. By satisfying Equations (3) and (4), the load amount can be increased as the rehabilitation progresses while ensuring the total load amount from the start time of rehabilitation to the end time of the rehabilitation.

The derivation unit 32 determines various types of partial load amounts Yai(tₐ) such that La(tₐ) satisfies Equations (3) and (4). Then, the derivation unit 32 derives the weight Wi(tₐ) using Equation (1) based on the partial load amount Yai(tₐ), the learning exercise information Xai(tₐ) and Ci.

Note that, in FIG. 4, various types of partial load amounts Yai(tₐ) are illustrated as straight lines that monotonically increase, but the present disclosure is not limited to this. For example, the partial load amount Yai(tₐ) may partially or entirely decrease over time, or may be a curve.

Further, the derivation unit 32 may use machine learning to derive the weight. For example, the partial load amount Yai(tₐ) may be derived by a trained model such as a convolutional neural network (CNN) or the like, which is trained in advance such that an input is various types of learning exercise information Xai(tₐ) and an output is various types of partial load amounts Yai(tₐ). Further, for example, a calculator may be used in which the partial load amount Yai(tₐ) derived from the trained model is used as an input and the weight Wi(tₐ) is used as an output.

Derivation of Load Amount

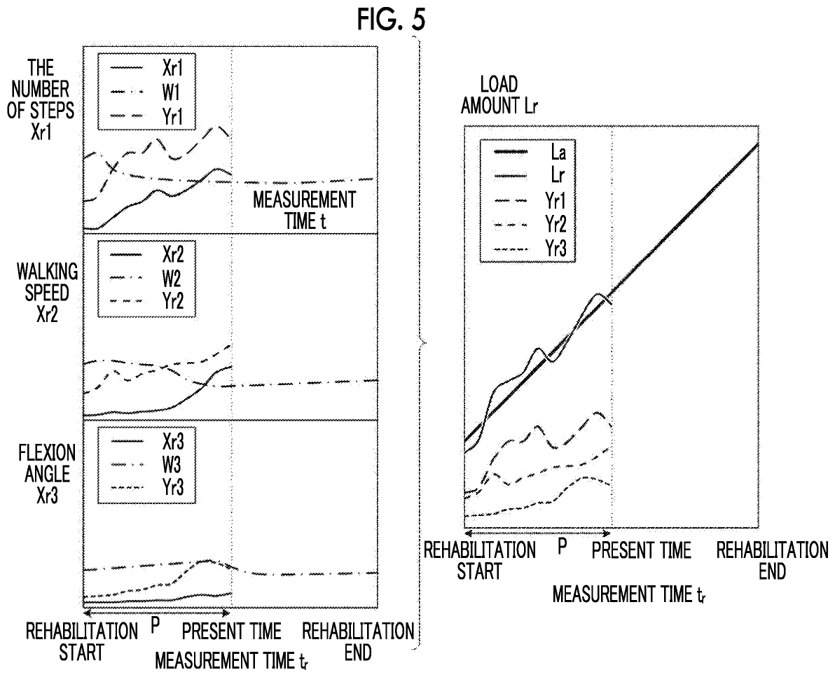

Next, with reference to FIG. 5, a method of deriving the load amount applied to the body of the subject by exercise will be described. The load amount is derived based on the exercise information and the weight. In FIG. 5, the number of steps Xr1, a walking speed Xr2, and a flexion angle Xr3 are indicated by solid lines. Further, weights W1, W2, and W3 for the number of steps Xr1, the walking speed Xr2, and the flexion angle Xr3 are indicated by one-dot chain lines. A partial load amount Yr1 related to the number of steps Xr1 is indicated by a long broken line, a partial load amount Yr2 related to the walking speed Xr2 is indicated by a short broken line, and a partial load amount Yr3 related to the flexion angle Xr3 is indicated by a dotted line. A load amount Lr is indicated by a thin solid line, and a reference load amount La (see FIG. 4) is indicated by a thick solid line.

The acquisition unit 30 acquires, from the exercise information measuring device 12, a plurality of pieces of exercise information Xri(tᵣ) indicating each of a plurality of types of exercise amounts measured according to the exercise of the subject. For example, the acquisition unit 30 acquires the number of steps Xr1, the walking speed Xr2, and the flexion angle Xr3 during a period P from a start time of rehabilitation to a present time.

The acquisition unit 30 acquires a weight Wi(tᵣ) derived in advance corresponding to the type of the acquired exercise information. Specifically, the acquisition unit 30 acquires the weight Wi(tₐ) derived at the measurement time tₐ of the learning exercise information corresponding to the measurement time tᵣ of the acquired exercise information, as the weight Wi(tᵣ). The weight Wi(tₐ) varies over time according to the measurement time tₐ of the learning exercise information Xai(tₐ) as described above. Therefore, the weight Wi(tᵣ) also varies over time according to the measurement time tᵣ of the exercise information.

Note that the acquisition unit 30 only needs to acquire the weight Wi(tᵣ) corresponding to the measurement time tᵣ of the acquired exercise information, and for example, may omit acquisition of the weight Wi(tᵣ) corresponding to a period after the present time at which the exercise information is not yet measured.

The derivation unit 32 derives the load amount Lr(tᵣ) applied to the body of the subject based on the plurality of pieces of exercise information Xri(tᵣ) and the weight Wi(tᵣ) acquired by the acquisition unit 30. The load amount Lr(tᵣ) is expressed by the following calculation equations (5) to (6).

Yri(tᵣ)=Wi(tᵣ)×Xri(tᵣ)×Ci (5)

Lr(tᵣ)=ΣYri(tᵣ) (6)

Note that i is an integer of 2 or more, and i is assumed to be 1 to 3 in the following description.

Yri(tᵣ) shown in Equation (5) represents the partial load amount applied to the body of the subject by the exercise amount indicated by the exercise information Xri(t) at the measurement time tᵣ. Ci is a conversion factor similar to that of Equation (1) described above. The derivation unit 32 derives Yri(tᵣ) using Equation (5) based on the plurality of pieces of exercise information Xri(t) and the weights Wi(tᵣ) acquired by the acquisition unit 30.

Lr(tᵣ) shown in Equation (6) is a sum of the partial load amounts Yri(tᵣ) at the measurement time tᵣ, and represents a total load amount applied to the body of the subject by the exercise amount indicated by various types of exercise information Xri(tᵣ) at the measurement time (tᵣ). The derivation unit 32 derives Lr(tᵣ) using Equation (6) based on Yri(tᵣ) derived using Equation (5). As described above, the load amount Lr(tᵣ) is derived by taking into account the weight Wi(tᵣ) which varies over time, and reflects the state of the physical function of the subject.

In practice, since various types of exercise information can be measured individually or the like, the measurement times tᵣ of the various types of exercise information do not necessarily match. Therefore, the derivation unit 32 may regard various types of exercise information measured within a predetermined period (for example, seven days) as being measured at the same measurement time tᵣ. That is, the derivation unit 32 may derive the load amount Lr(tᵣ) based on a plurality of pieces of exercise information Xri(tᵣ) measured within the predetermined period (for example, seven days).

Prediction of Biological Information at Prediction Timing

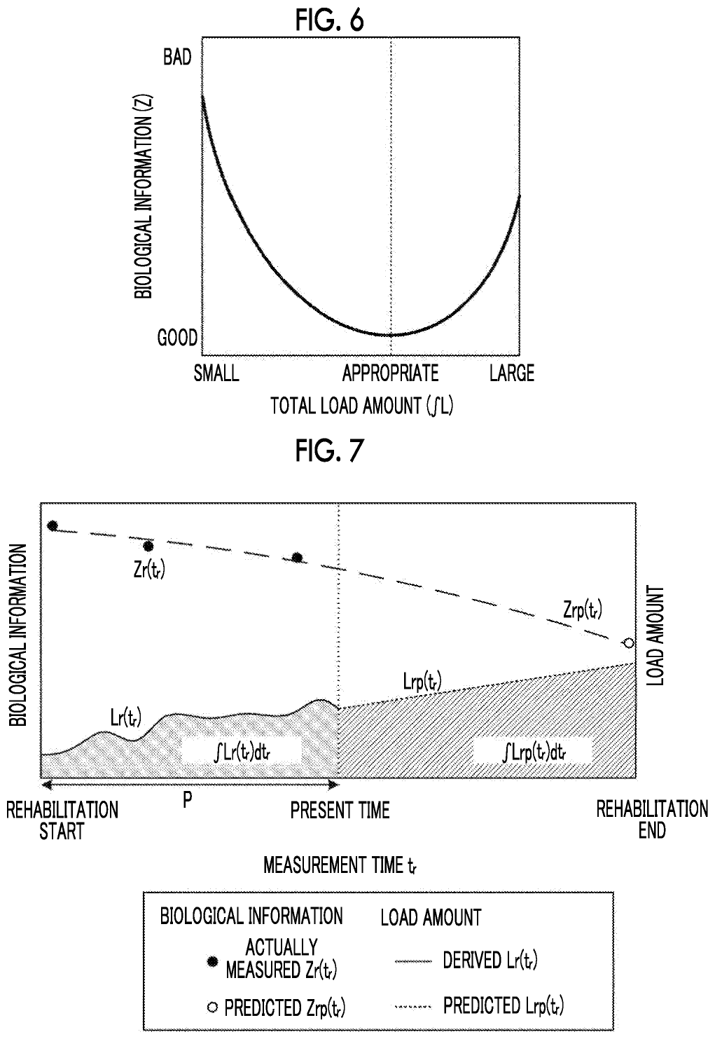

Next, with reference to FIGS. 6 and 7, a method of predicting biological information (hereinafter referred to as “prediction biological information”) at a prediction timing will be described.

The acquisition unit 30 acquires biological information measured about the subject from the biological information measuring device 14. In addition, the acquisition unit 30 acquires the load amount applied to the body of the subject by the exercise that affects the biological information performed within a predetermined period up to the present time. For example, the acquisition unit 30 acquires the load amount Lr(tᵣ) based on the exercise information Xri(tᵣ) and the weight Wi(tᵣ) derived by the derivation unit 32 in the period P from the start time of the rehabilitation to the present time.

It is known that the biological information is improved or deteriorated according to the total load amount from the start time to the end time of the rehabilitation (that is, the time integral ∫Lr(tᵣ)dtᵣ of the load amount Lr(tᵣ)). FIG. 6 is a correlation diagram showing an example of the correlation between the biological information and the total load amount. The correlation between the biological information and the total load amount is determined in advance for each type of the biological information and stored in advance, for example, in the storage unit 22.

In the case of a defect volume of the knee cartilage as an example of the biological information, it is known that, as shown in FIG. 6, the defect volume increases (deteriorates) in a case where the total load amount is excessive or too small, and the defect volume decreases (improves) in a case where the total load amount is appropriate. In cartilage regeneration by mesenchymal stem cell transplantation, by transplanting mesenchymal stem cells into a cartilage defect, the transplanted mesenchymal stem cells differentiate to produce chondroblasts, the produced chondroblasts produce chondrocytes, and the produced chondrocytes produce cartilage (specifically, a matrix of collagen and proteoglycan). Thereby, the cartilage defect is repaired. In order to promote differentiation and proliferation of each of the cells and promote the repair of the cartilage defect, rehabilitation is necessary, and in particular, it is important to implement rehabilitation in which the exercise amount is appropriately controlled according to the state of repair. Insufficient rehabilitation may not promote the repair sufficiently, and excessive rehabilitation may destroy and reduce each cell in the process of the repair.

On the other hand, a time lag may occur until the effect of exercise training in rehabilitation is reflected in the biological information. Therefore, the prediction unit 34 predicts prediction biological information at a predetermined prediction timing after the present time based on the biological information and the load amount acquired by the acquisition unit 30. FIG. 7 is a graph showing an actually measured value Zr(tᵣ) of the biological information and a derived value of the load amount Lr(tᵣ) in the period P from the start time of the rehabilitation to the present time, and a predicted value Zrp(tᵣ) of the biological information and a predicted value Lrp(tᵣ) of the load amount in the period after the present time.

Specifically, the prediction unit 34 may use the learning information DB 16 to search for past cases similar to the biological information and the total load amount up to the present time, and may use the learning biological information in the past cases to predict the prediction biological information. Further, the prediction unit 34 may predict the prediction biological information using a trained model such as a CNN, which is trained in advance such that the input is the biological information and the load amount and the output is the prediction biological information.

The prediction timing may be, for example, a timing after a predetermined period (for example, one month) has elapsed from the present time, or a timing optionally set by the user via the input unit 25. That is, the prediction unit 34 may receive designation of the prediction timing by the user.

Further, for example, the prediction unit 34 may receive designation of a variation target related to the biological information, and set a timing at which the biological information can achieve the variation target (for example, a shortest timing or a standard timing) as the prediction timing. Specifically, the prediction unit 34 may predict an expiration time of the period necessary for the biological information to achieve the variation target based on the biological information and the load amount acquired by the acquisition unit 30, and set the expiration time as the prediction timing. For example, the prediction unit 34 may use the learning information DB 16 to search for past cases similar to the biological information and the load amount up to the present time, and specify a time when the learning biological information achieves the variation target in the past cases to predict the expiration time. The variation target related to the biological information may be designated by a numerical value such as, for example, a 20% reduction in defect volume of the knee cartilage.

Plan for Exercise Training

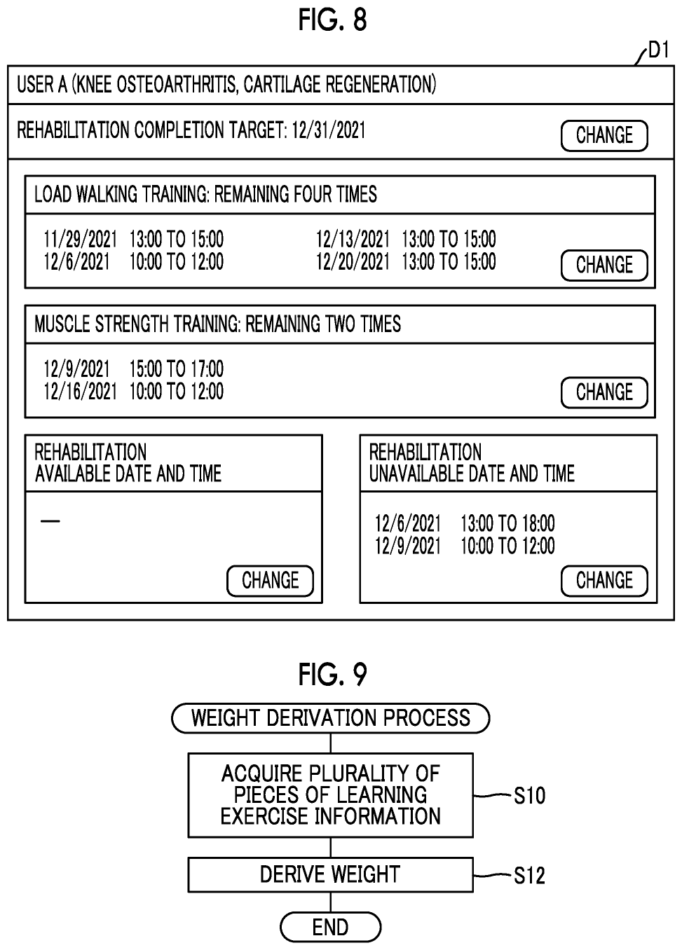

Next, a method of planning exercise training in rehabilitation will be described with reference to FIGS. 7 and 8. As described above, a time lag may occur until the effect of exercise training in rehabilitation is reflected in the biological information. Therefore, in order to match the biological information that can be measured at the prediction timing with the prediction biological information, it is required to perform exercise training in consideration of the time lag.

The planning unit 36 creates a plan related to exercise necessary to match the biological information that can be measured at the prediction timing with the prediction biological information predicted by the prediction unit 34. FIG. 8 is an example of a screen D1 displayed on the display 24 and including exercise training planned by the planning unit 36.

Specifically, the planning unit 36 derives a necessary load amount, which is a load amount applied to the body of the subject necessary to match the biological information that can be measured at the prediction timing with the prediction biological information. For example, the planning unit 36 searches for similar past cases using the learning information DB 16 based on the biological information Zr(tᵣ) and the load amount Lr(tᵣ)up to the present time acquired by the acquisition unit 30. Further, the planning unit 36 specifies a total load amount ∫La(tₐ)dtₐ required until learning biological information Za(tₐ) corresponding to the current biological information Zr(tᵣ) varies to learning biological information Zap(tₐ) corresponding to the prediction biological information Zrp(tᵣ) in the past case. Then, the planning unit 36 derives a difference between the specified total load amount ∫La(tₐ)dtₐ and the total load amount ∫Lr(tᵣ)dtᵣ up to the present time derived by the derivation unit 32, as the necessary load amount ∫Lrp(tᵣ)dtᵣ.

Further, for example, the planning unit 36 may derive the necessary load amount using a trained model such as a CNN that has been trained in advance such that the input is the current biological information Zr and the prediction biological information Zrp and the output is the necessary load amount.

In addition, the planning unit 36 creates a plan related to the exercise satisfying the derived necessary load amount. Specifically, as shown in FIG. 8, the planning unit 36 creates a plan in which a type of the exercise to be performed is defined among a plurality of types of exercise having different loads. For example, a type of effective exercise training differs between the early stage and the late stage of the rehabilitation. Therefore, the planning unit 36 creates a plan in which the content of the exercise training suitable for varying the current biological information Zr to the prediction biological information Zrp is defined. The load for each exercise training is stored in advance, for example, in the storage unit 22.

In addition, as shown in FIG. 8, the planning unit 36 creates a plan that defines a schedule for performing exercise necessary to match the biological information that is measurable at the prediction timing with the prediction biological information Zrp. For example, the planning unit 36 may create a plan in consideration of the convenience of the subject, the convenience of the institution for performing the rehabilitation, and the like. Specifically, the planning unit 36 may receive designation of at least one of a date and time at which the subject is able to perform exercise or a date and time at which the subject is not able to perform exercise, and create the plan according to the received designation of the date and time.

The controller 38 performs control to display the screen D1 including the plan for exercise training in the rehabilitation created by the planning unit 36 on the display 24.

Next, an operation of the information processing device 10 according to the present embodiment will be described with reference to FIGS. 9 to 12. In the information processing device 10, the CPU 21 executes the information processing program 27 to execute a weight derivation process, a load amount derivation process, a prediction process, and a planning process shown in FIGS. 9 to 12. For example, in a case where the user gives an instruction to start execution of the process via the input unit 25, the respective processes may be executed in succession in the order of the weight derivation process, the load amount derivation process, the prediction process, and the planning process, or only the designated process may be executed individually.

First, the weight derivation process will be described with reference to FIG. 9. In step S10, the acquisition unit 30 acquires a plurality of pieces of the learning exercise information from the learning information DB 16. In step S12, the derivation unit 32 derives a weight for each piece of the learning exercise information based on the plurality of pieces of the learning exercise information acquired in step S10 and ends the weight derivation process.

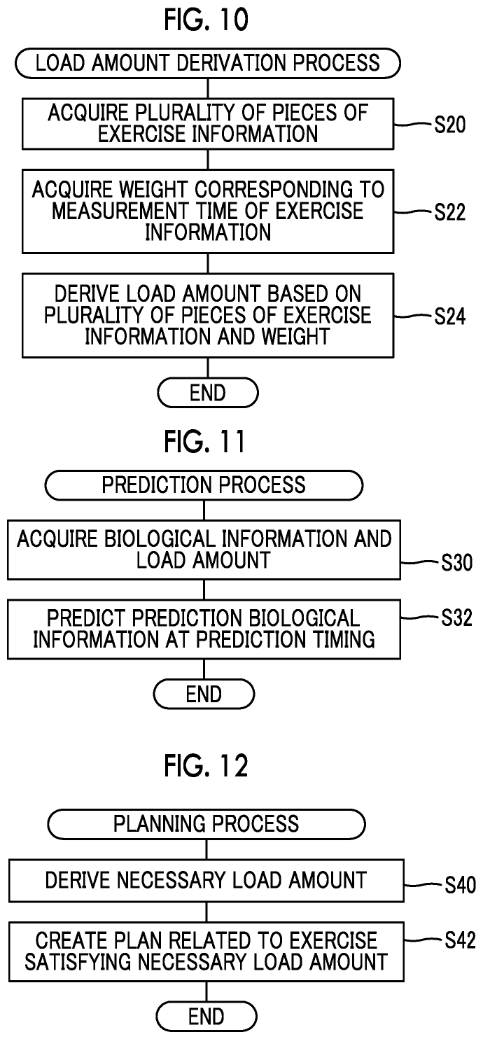

Next, the load amount derivation process will be described with reference to FIG. 10. In step S20, the acquisition unit 30 acquires a plurality of pieces of the exercise information from the exercise information measuring device 12. In step S22, the acquisition unit 30 acquires the weight derived in advance corresponding to the type of the exercise information acquired in step S20 (that is, the weight derived in step S12). In step S24, the derivation unit 32 derives the load amount applied to the body of the subject based on the plurality of pieces of the exercise information acquired in step S20 and the weight acquired in step S22 and ends the load amount derivation process.

Next, a prediction process of the prediction biological information at the prediction timing will be described with reference to FIG. 11. In step S30, the acquisition unit 30 acquires the biological information from the biological information measuring device 14. Further, the acquisition unit 30 acquires the load amount applied to the body of the subject (that is, the load amount derived in step S24). In step S32, the prediction unit 34 predicts the prediction biological information at a predetermined prediction timing after the present time based on the biological information and the load amount acquired in step S30 and ends the prediction process.

Next, the planning process of the exercise training in rehabilitation will be described with reference to FIG. 12. In step S40, the planning unit 36 derives the necessary load amount for matching the biological information that is measurable at the prediction timing with the prediction biological information (that is, the prediction biological information predicted in step S32). In step S42, the planning unit 36 creates a plan related to exercise training that satisfies the necessary load amount derived in step S40 and ends the planning process.

As described above, the information processing device 10 according to one aspect of the present disclosure acquires a plurality of pieces of exercise information indicating each of a plurality of types of exercise amounts measured according to the exercise of the subject, acquires a weight derived in advance based on the exercise information for each type of the exercise information, and derives a load amount applied to the body of the subject based on the plurality of pieces of exercise information and the weight.

That is, with the information processing device 10 according to the present embodiment, the load amount corresponding to the state of the physical function of the subject can be derived by using the weight derived based on the exercise information. Therefore, the load amount applied to the body of the subject by exercise training up to the present time and the load amount applied to the body of the subject by exercise training to be performed from now on can be derived as indices in which the state of the physical function of the subject is taken into consideration, so that it is possible to support the improvement of the physical function.

In addition, the information processing device 10 according to another aspect of the present disclosure acquires biological information measured about a subject and a load amount applied to a body of the subject by exercise that affects the biological information performed within a predetermined period up to a present time, predicts prediction biological information, which is the biological information at a predetermined prediction timing after the present time, based on the biological information and the load amount, and creates a plan related to exercise necessary to match the biological information that is measurable at the prediction timing with the prediction biological information.

That is, with the information processing device 10 according to the present embodiment, the prediction biological information at the prediction timing can be predicted based on the biological information and the load amount up to the present time, and the exercise training can be planned by setting the prediction biological information as a target, so that it is possible to support the improvement of the physical function. Furthermore, by using the load amount derived in consideration of the state of the physical function of the subject, a plan can be created in consideration of the effects of various types of exercise training that vary according to the state of the physical function of the subject, so that it is possible to further support the improvement of the physical function.

In the above-described embodiment, the embodiment in which the load amount according to the state of the physical function of the subject is derived by varying the weight according to a time axis from the start time to the end time of the rehabilitation, but the present disclosure is not limited to this. More precisely, it is preferable that the acquisition unit 30 acquires the physical information indicating the physical level of the subject, and the derivation unit 32 derives the load amount reflecting the physical level (physical function of the subject) by varying the weight according to the physical information.

As a premise in this case, physical information indicating the physical level of the subject being a measurement source at the measurement time may be added to the learning exercise information and the learning biological information stored in the learning information DB 16. The derivation unit 32 may derive the weight based on the learning exercise information to which the physical information corresponding to the physical information added to the exercise information acquired from the exercise information measuring device 12 is added. That is, the derivation unit 32 may derive the weight based on learning exercise information measured from a subject having a physical level equivalent to that of the subject to be currently processed.

The physical level may indicate, for example, an improvement level in a case of improving the physical function of the subject. The improvement level is represented by, for example, objective evaluations such as a maximum walking speed and a range of motion of a joint, and subjective evaluations such as a degree of pain due to exercise.

In addition, the planning unit 36 may create a plan defining that a predetermined type of exercise is to be performed according to the physical information indicating the physical level of the subject. For example, the planning unit 36 may vary the type of exercise training to be performed according to the range of motion of the knee joint of a patient with knee osteoarthritis.

In the above-described embodiment, the derivation unit 32 may derive, as the load amount, the load amount applied to a part of the body of the subject such as a knee joint and a hip joint. For example, effective exercise training for rehabilitation differs between the knee joint and the hip joint. Therefore, for example, the derivation unit 32 may derive the load amount applied to the knee joint and the load amount applied to the hip joint using different types of exercise information and weights.

Further, in the above-described embodiment, the support for rehabilitation performed for improving deterioration in physical function in the treatment of knee osteoarthritis has been described, but the technique of the present disclosure can also be applied to other cases. The technique of the present disclosure may be applied to the support for rehabilitation performed for improving deterioration in physical function due to, for example, trauma, aging, brain dysfunction, and the like. For example, in order to suppress the deterioration in intellectual functions such as understanding, judgment and logic as in dementia, the present disclosure may be applied to rehabilitation that includes not only exercise training related to the body but also training of intellectual functions such as calculation and puzzles. The load amount in this case may be derived using, for example, a correct rate of calculation, a time required to complete puzzles, and the like. Further, for example, the present disclosure may be applied to the support for muscle strength training performed to improve the physical functions of a healthy person and the like. That is, the “physical level” is not limited to the improvement level in a case of improving the physical function of the subject, but may be a muscle strength level of the subject and the like. In addition, depending on each case, types of a plurality of pieces of exercise information and biological information used for processing and their combinations may be appropriately changed.

Further, in the above embodiment, for example, as a hardware structure of a processing unit that executes various types of processing such as the acquisition unit 30, the derivation unit 32, the prediction unit 34, the planning unit 36, and the controller 38, various types of processors shown below can be used. The various types of processors include, as described above, a CPU which is a general-purpose processor that executes software (program) to function as various types of processing units, as well as a programmable logic device (PLD) which is a processor having a circuit configuration that can be changed after manufacturing such as a field programmable gate array (FPGA), a dedicated electrical circuit which is a processor having a circuit configuration specially designed to execute specific processing such as an application specific integrated circuit (ASIC), and the like.

One processing unit may be configured of one of the various types of processors, or a combination of two or more processors of the same type or different types (for example, a combination of a plurality of FPGAs, or a combination of a CPU and an FPGA). Further, a plurality of processing units may be configured of one processor.

As an example of configuring a plurality of processing units with one processor, first, there is a form in which, as typified by computers such as a client and a server, one processor is configured by combining one or more CPUs and software, and the processor functions as a plurality of processing units. Second, there is a form in which, as typified by a system on chip (SoC) and the like, in which a processor that implements functions of an entire system including a plurality of processing units with one integrated circuit (IC) chip is used. As described above, the various types of processing units are configured using one or more of the various types of processors as a hardware structure.

Furthermore, as the hardware structure of the various types of processors, more specifically, an electric circuitry in which circuit elements such as semiconductor elements are combined can be used.

Further, in the above-described embodiment, an aspect in which the information processing program 27 is stored (installed) in advance in the storage unit 22, but the present disclosure is not limited to this. The information processing program 27 may be provided in a form recorded in a recording medium such as a compact disc read only memory (CD-ROM), a digital versatile disc read only memory (DVD-ROM), and a universal serial bus (USB) memory, or may be provided in a form stored in a large-scale database on a cloud. Further, the information processing program 27 may be downloaded from an external device via a network. Furthermore, in addition to the information processing program, the technique of the present disclosure extends to a storage medium that non-transitorily stores the information processing program.

The technique of the present disclosure can also appropriately combine the above-described embodiments. The above-descried contents and illustrated contents are detailed descriptions of parts related to the technique of the present disclosure, and are merely examples of the technique of the present disclosure. For example, the above descriptions related to configurations, functions, operations, and effects are descriptions related to examples of configurations, functions, operations, and effects of the parts related to the technique of the present disclosure. Therefore, it is needless to say that unnecessary parts may be deleted, or new elements may be added or replaced with respect to the above-described contents and illustrated contents within a scope not departing from the spirit of the technique of the present disclosure.

What is claimed is:

1. An information processing device comprising at least one processor, wherein the at least one processor is configured to:
acquire a plurality of pieces of exercise information indicating each of a plurality of types of exercise amounts measured according to exercise of a subject;
acquire a weight derived in advance based on the exercise information for each type of the exercise information; and
derive a load amount applied to a body of the subject based on the plurality of pieces of exercise information and the weight.

2. The information processing device according to claim 1, wherein:
the plurality of pieces of exercise information are information indicating each of a plurality of types of exercise amounts measured over time, and
the at least one processor is configured to acquire the weight that varies over time according to a measurement time of the exercise information.

3. The information processing device according to claim 2, wherein the at least one processor is configured to:
acquire a plurality of pieces of learning exercise information that are of the same type as the plurality of pieces of exercise information and that are measured over time;
derive the weight that varies over time according to a measurement time of the learning exercise information based on the plurality of pieces of learning exercise information; and
derive the load amount based on the weight derived at the measurement time of the learning exercise information corresponding to the measurement time of the exercise information.

4. The information processing device according to claim 3, wherein:
physical information indicating a physical level of the subject being a measurement source at the measurement time is added to the exercise information and the learning exercise information, and
the at least one processor is configured to derive the weight based on the learning exercise information to which the physical information corresponding to the physical information added to the exercise information is added.

5. The information processing device according to claim 4, wherein the physical level indicates an improvement level in a case of improving a physical function of the subject.

6. The information processing device according to claim 1, wherein the at least one processor is configured to derive a load amount applied to a part of the body of the subject as the load amount.

7. The information processing device according to claim 1, wherein the at least one processor is configured to derive the load amount based on the plurality of pieces of exercise information measured within a predetermined period.

8. The information processing device according to claim 1, wherein the exercise information indicates at least one of the number of steps, a walking speed, an electromyogram, or a flexion angle or a flexion speed of a joint of the subject.

9. An information processing method comprising:
acquiring a plurality of pieces of exercise information indicating each of a plurality of types of exercise amounts measured according to exercise of a subject;
acquiring a weight derived in advance based on the exercise information for each type of the exercise information; and
deriving a load amount applied to a body of the subject based on the plurality of pieces of exercise information and the weight.

10. A non-transitory computer-readable storage medium storing an information processing program for causing a computer to execute a process comprising:
acquiring a plurality of pieces of exercise information indicating each of a plurality of types of exercise amounts measured according to exercise of a subject;
acquiring a weight derived in advance based on the exercise information for each type of the exercise information; and
deriving a load amount applied to a body of the subject based on the plurality of pieces of exercise information and the weight.
